SEQUENCE LISTING (18 sequences)

SEQ ID NO: 1 - protein, 538 aa, synthetic construct
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 2 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 3 - protein, 617 aa, synthetic construct
MVRPWVFCLLLFPCSSAYSDSWMPLVNLTQHLLQEANSSFSSNCWVCLSIQTQRSLAMPAPLRTWTETPMKLRIMYSARTLSGPYPITDLERRLQNFQPLTPHSSFVNPDQRAIAFLQITSVTGILPILSRITSVRYPDDHVYESAQRPIWGSLSTQTILTSQAPLCISRFFKNSNHATFVGKLPASLCNHTFQLSPSANHQSIDLSSSYAFAPLMAMPGSKWRNPLRFSGPPSLNSGMPHYSCPIDDIHCHTYPTTPWRSCPSFPASTCYNLTLFEPDNSSHPITLSVDTTYFKIKLQGHKDPYPLFQYQPLMGAALSGQYSIWEYEPTVKKNGGITPNIFSHLVSLTYSFCLNSSGVFFLCGNSTYVCLPANWSGVCTLVFQYPDIELLPNNQTISVPLFATVPSSVPASRRKRALPLLPLLAGLGIASALGLGIAGITTSTVYFQQLSKALSDSLDEIATSIISLQDQIDSLAGVVLQNRRALDLIVAERGGTCLFLQEECCFYINQSGVVRHAARKLRERASELGTSSSSWIQWLGLGPWLPSWLTSLMAPILFILVLLVFRPCLLNCLTHSVSRRMSSFIHTTTEGHVDKILLLRESQYKRLPQEPPEEDAV

SEQ ID NO: 4 - protein, 494 aa, synthetic construct
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLY

SEQ ID NO: 5 - protein, 396 aa, synthetic construct
MELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 6 - protein, 240 aa, synthetic construct
LQQSGGGLVQPGGSMKLSCVASGFTSSNYWLNWVRQSPERGLEWVAEIRLKSNNYATNYAESVKGRFTISRDDSKSSVYLQVNNLRAEDTGIYYCTRSTARATPYYFDYWGQGTTVTGGGGSGGGGSGGGGSELTQSPSSLAVSAGEKVTMSCKSSQSLLNSRARKNFLTWYQQKPGQSPKLLIYWASTRESGVPDRFTGSGSGTDFTLTISSVQAEDLAVYYCKQSYNLYTFGGGTKLE

SEQ ID NO: 7 - protein, 246 aa, synthetic construct
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEIKFREQ

SEQ ID NO: 8 - protein, 30 aa, synthetic construct
WEARLARALARALARHLARALARALRACEA

SEQ ID NO: 9 - protein, 21 aa, synthetic construct
  MOD_RES 10: note = Orn
  MOD_RES 11: note = Orn
  MOD_RES 18: note = Orn
  MOD_RES 19: note = Orn
AGYLLGKLLXXLAAAALXXLL

SEQ ID NO: 10 - protein, 6 aa, synthetic construct
PFVYLI

SEQ ID NO: 11 - protein, 27 aa, synthetic construct
WEAKLAKALAKALAKHLAKALAKALKA

SEQ ID NO: 12 - protein, 30 aa, synthetic construct
WEAALAEALAEALAEHLAEALAEALEALAA

SEQ ID NO: 13 - protein, 26 aa, synthetic construct
KKALLAHALHLLALLALHLAHALKKA

SEQ ID NO: 14 - protein, 21 aa, synthetic construct
KLLLLKLLLLKLLLLKLLLLK

SEQ ID NO: 15 - protein, 15 aa, synthetic construct
  MOD_RES 3: note = Aib
  MOD_RES 6: note = Aib
  MOD_RES 9: note = Aib
  MOD_RES 12: note = Aib
  MOD_RES 15: note = Aib
RRXRRXRRXRRXRRX

SEQ ID NO: 16 - protein, 963 aa, synthetic construct
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGSGEGRGSLLTCGDVEENPGPMEVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSGGGGSGGGGSGGGGSEIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEIKFREQGSGATNFSLLKQAGDVEENPGPMALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 17 - protein, 957 aa, synthetic construct
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGSGEGRGSLLTCGDVEENPGPMLQQSGGGLVQPGGSMKLSCVASGFTSSNYWLNWVRQSPERGLEWVAEIRLKSNNYATNYAESVKGRFTISRDDSKSSVYLQVNNLRAEDTGIYYCTRSTARATPYYFDYWGQGTTVTGGGGSGGGGSGGGGSELTQSPSSLAVSAGEKVTMSCKSSQSLLNSRARKNFLTWYQQKPGQSPKLLIYWASTRESGVPDRFTGSGSGTDFTLTISSVQAEDLAVYYCKQSYNLYTFGGGTKLEGSGATNFSLLKQAGDVEENPGPMALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 18 - DNA, 7690 bp, synthetic construct
  source 1..7690: mol_type = other DNA
atcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcgccaccatggccctgccctatcacatcttcctcttcacggtgctgctgccgtcattcacactcaccgcgccgcccccctgccggtgcatgacaagctccagtccctaccaagagttcctgtggcgcatgcaaaggcctgggaacatcgacgccccttcttacaggtccttaagcaagggcacaccaaccttcaccgctcacacccacatgcctcgtaactgctatcacagtgctacactttgcatgcatgcaaacacccattactggactggcaagatgatcaaccctagttgccccggcggcctgggagttaccgtgtgctggacgtactttacacagacaggaatgtctgacggcggcggggtacaagaccaagcgagagagaagcatgtaaaggaggttatatcccagctgaccagagttcacggcacatccagcccctataagggactggacttaagcaaacttcacgagaccctccgcacacatactagattggtctcactgttcaatacaactctgacaggactgcacgaggtctcagcccagaaccccactaactgttggatttgcttaccactgaatttcaggccctatgtgtctatccccgtcccggaacagtggaataacttcagcaccgagattaacactactagcgttcttgtaggtcctttggtcagcaatctggagatcacacatacctctaatctcacatgcgtgaagttctcaaatacaacatacacgactaacagccagtgtattcggtgggtcacccccccgacccagatcgtgtgcctgccttccggaattttttttgtgtgcggcacatcagcttaccggtgtctgaacggcagttccgagagcatgtgtttcttgagttttctggtccctcctatgaccatttacacagagcaagatctgtactcctatgttatcagcaagccaagaaataaacgggttcccattcttcctttcgtcatcggggccggtgtccttggagctcttgggaccgggatcggaggcatcactaccagcacacagttttattataaactcagtcaggagctcaacggcgatatggaaagagtggctgacagcttagttacactgcaggatcagttaaattccctggccgccgtggtgctgcagaatagaagggcactggatctgctgaccgccgagcggggcggcacctgcttgtttctcggcgaggaatgctgttattacgtgaaccagtcaggtatagttactgagaaggtcaaggagatccgtgatcgaattcagaggagggccgaagaactgaggaacactgggccatggggactgctgtctcagtggatgccgtggatacttcccttcttaggaccccttgcagcaattatccttttgttactgtttggaccttgcatctttaaccttctggtgaattttgtgtcttccaggattgaggccgtcaagctgcagatggagccaaaaatgcaatctaaaactaagatttaccggcggcccctggatcgcccagccagcccgaggagtgatgtgaatgacattaagggcacaccacctgaggagattagcgcagctcagcctttgttgcggccaaacagtgcagggtccagttgaggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgt

(19) United States
(12) Patent Application Publication
(10) Pub. No.: US 2026/0295076 A1
(43) Pub. Date: Oct. 1, 2026

(54) CELLULAR DELIVERY OF THERAPEUTICS USING FUSOGENIC VESICLES

(71) Applicant: University of Maryland, Baltimore, Baltimore, MD (US)

(72) Inventors: Tim LUETKENS, Baltimore, MD (US); Aneesh KARATT VELLATT, Cambridge, Cambridgeshire (GB); Andrea MARTOS ESTEBAN, Cambridge, Cambridgeshire (GB)

(73) Assignee: University of Maryland, Baltimore, Baltimore, MD (US)

(21) Appl. No.: 19/480,569

(22) PCT Filed: May 2, 2024
(86) PCT No.: PCT/US2024/027464

§ 371 Date: Oct. 31, 2025

Related U.S. Application Data
(60) Provisional application No. 63/463,370, filed on May 2, 2023.
(60) Provisional application No. 63/524,312, filed on Jun. 30, 2023.

(51) Int. Cl. A61K 48/00; A61K 9/127; A61K 38/46

(52) U.S. Cl. CPC A61K 48/0033; A61K 9/127; A61K 38/465; A61K 48/005

(57) ABSTRACT

Engineered cells that serve as vehicles to deliver genetically-encoded cargo molecules, such as therapeutic molecules, to other cells in vivo are provided. In particular, fusogenic biovesicles with reduced immunogenicity potential, expressing poorly immunogenic (fully human or humanised or deimmunised) fusogens enclosing the cargo molecules are produced by the engineered cells. The engineered cells can be used in methods of treating diseases and conditions such as amyotrophic lateral sclerosis, Alzheimer's disease and cancer.

### STATEMENT OF FEDERALLY SPONSORED RESEARCH AND DEVELOPMENT

This invention was made with government support under Grant Number HT9425-23-1-0195 awarded by the US Army Medical Research Development and Command. The government has certain rights in the invention.

### SEQUENCE LISTING

A sequence listing in electronic (XML file) format is filed with this application and incorporated herein by reference. The name of the XML file is “Sequence_Listing_2024-0549A.xml”; the file was created on May 2, 2024; the size of the file is 32,782 bytes.

### BACKGROUND OF INVENTION

Adoptive cell therapy, in particular using immune cells, has seen great progress in recent years. The advances in the genetic engineering of immune cells, including safe and effective retroviral delivery and transposon-based systems, such as Sleeping Beauty, and the comprehensive characterization of transgenic T cell function in hundreds of clinical trials, have revolutionized the field of cancer immunotherapy [2]. These advances are at least in part the result of these cells' exquisite specificity and prolonged action due to their persistence in the patient's body. However, these advances have failed to similarly benefit other areas of major biomedical need, such as neurodegenerative and other chronic diseases. This can be attributed to qualitative differences in the subcellular localization of the respective molecular targets (surface vs. intracellular antigens) as well as the required mechanism of action (target cell killing vs. protein degradation/inhibition). Therefore, approaches that can facilitate the delivery and release of cargo, such as therapeutic molecules, to the cytosol of target cells have broad applicability for the treatment of a multitude of diseases [30].

Biovesicles (BVs) represent a range of extracellular vesicles (EV) including exosomes, microvesicles and virus like particles (VLPs) with the ability to encapsulate and be released as cargo-containing vesicles. High levels of BVs are tolerated by the human host as these are naturally generated in the body and can deliver a wide variety of cargo such as small molecules, proteins, mRNAs and microRNAs. Despite vast possibilities and recent clinical advances, the development of both EV- and BV-based therapeutics, has also proven to be challenging especially for the treatment of chronic diseases. This is driven by difficulties in establishing scalable and reproducible ex vivo manufacturing processes, the sorting mechanism of source cell molecules into EVs, low effective release of cargo into the target cell, and potential need for repeated dosing. In addition, to date the use of BVs for therapy has been limited to in vitro production and subsequent administration.

Mangeot et al. had devised a system for in vitro production of fusogenic vesicles (gesicles) by overexpression of the spike glycoprotein of the vesicular stomatitis virus (VSV-G) in HEK cells [31]. However, the use of this system is unlikely to be suitable for in vivo production of therapeutic BVs or for repeated administration of in vitro produced EV to deliver cargo due to the immunogenicity of VSV-G.

In summary, currently no strategies exist to generate cells stably producing BVs to deliver a wide range of cargo in a persistent and targeted manner with sufficient efficiency to modulate disease processes. It is also unknown whether such cells can be engineered efficiently in a scalable manner.

The present invention is directed to overcoming these hurdles and other important goals in the development of effective means for intracellular delivery of cargo, such as therapeutic molecules, to target cells.

### BRIEF SUMMARY OF INVENTION

The present invention is broadly directed to engineered cells that serve as a source of biovesicles (BVs) that can deliver cargo molecules, such as therapeutic molecules, to other cells in vivo or in vitro. In an in vivo setting, cargo molecules are shuttled to target cells within cell-derived fusogenic BVs, generated by the engineered source cell administered in vivo. In an in vitro setting, such engineered cells serve as a source for production or manufacture of the cell-derived fusogenic BVs that can then be used in subsequent therapeutic administration. Encapsulated cargo molecules include, but are not limited to, protein therapeutics as well as mRNA that encodes protein therapeutics that will be translated in target cells. This invention can be applied to therapeutic applications (such as diseased cells or healthy cells acting as carriers) using therapeutic molecules as the cargo molecules and/or non-therapeutic applications (such as cell-based assays) using non-therapeutic molecules as the cargo molecules.

In an in vivo setting, similar to T cells expressing chimeric antigen receptors [2], these cargo molecule-carrying, fusogenic BV-producing engineered cells will persist in patients over extended periods of time, acting as a constant source of the respective protein therapeutic, for example. Such fusogenic BV-producing adoptive cell therapy (FACT) combines durability of a cell therapy with the functional flexibility of EVs to deliver in vivo-produced payloads from engineered source cells to target receiving cells. The FACT platform for the delivery of genetically encoded therapeutics by engineered cells represents a paradigm-shifting technology, potentially allowing for the treatment of many diseases and conditions.

The use of BVs enables delivery of cargos, such as drugs, to intracellular, transmembrane, and secreted targets. In particular, fusogenic BVs have the capacity to incorporate cargo molecules from a source cell, bud from the cell membrane of the source cell to the extracellular space and, upon fusing to the target cell membrane, release their cargo to the cytosol of the target cell. Specifically, in vivo generated fusogenic BVs comprising one or more therapeutic molecule(s) of interest as the cargo, such as a protein, can be used as protein replacement therapy to substitute absent or dysfunctional proteins or to deliver a protein or peptide therapeutic to modulate aberrant cellular pathways.

In a first embodiment, the present invention is directed to engineering FACT (fusogenic biovesicle-producing adoptive cell therapy) cells comprising (i) a polynucleotide sequence encoding one or more fusogens and/or proteins or peptides with cell penetrating properties, (ii) a polynucleotide sequence serving as or encoding one or more cargo molecules, and (iii) optionally a polynucleotide sequence encoding one or more gag-like proteins.

Each of the noted polynucleotide sequences can, independently of the other polynucleotide sequences, be a genomic (chromosomal) or non-genomic sequence. Non-genomic sequences are extra-chromosomal sequences such as those of a plasmid or virus present in the FACT cells.

Suitable cargo molecules include, but are not limited to, DNA molecules, RNA molecules, peptides and proteins. Exemplary peptides and proteins include, but are not limited to, enzymes such as gene-editing enzymes, including base editors, prime editors, TALE nucleases, and CRISPR/Cas, zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

The FACT cells of the invention produce and release fusogenic BVs comprising the one or more cargo molecules and the optional one or more gag-like proteins. In certain aspects of this embodiment, the cargo molecules are therapeutic molecules.

In a second embodiment, the invention is directed to fusogenic BVs comprising (i) one or more fusogens and/or proteins with cell penetrating properties, (ii) one or more cargo molecules, and (iii) optionally one or more gag-like proteins. It should be understood that the fusogenic BVs themselves are constructed of (i) the fusogens or proteins with cell penetrating properties, and (ii) the gag-like proteins, when present, to form a structure. The cargo molecules are present inside of the structure and thus carried within the fusogenic BVs. In certain aspects of this embodiment, the cargo molecules are therapeutic molecules. Suitable therapeutic molecules include, but are not limited to, DNA molecules, RNA molecules, peptides and proteins. Exemplary peptides and proteins include, but are not limited to, enzymes such as gene-editing enzymes, including base editors, prime editors, TALE nucleases, and CRISPR/Cas,zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function. Such genetically-encoded therapeutics can be used in the treatment of selected diseases and conditions.

In a third embodiment, the invention is directed to methods for delivering one or more cargo molecules to a target cell, comprising culturing a target cell with a fusogenic BV of the present invention under conditions promoting delivery of a cargo molecule from the fusogenic BV to the target cell. Such delivery is via binding of the fusogenic BVs to the target cell and release of the cargo molecules into the target cell. In certain aspects of this embodiment, the cargo molecules are therapeutic molecules. The therapeutic molecules carried by the fusogenic BVs may be, but are not limited to, DNA molecules, RNA molecules, peptides or proteins that are therapeutic for a selected disease or condition. Exemplary peptides and proteins include, but are not limited to, enzymes such as gene-editing enzymes, including base editors, prime editors, TALE nucleases, and CRISPR/Cas, zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

In a fourth embodiment, the invention is directed to methods for delivering one or more cargo molecules to a target cell, comprising culturing a target cell with a FACT cell of the present invention under conditions promoting delivery of a cargo molecule from the FACT cell to the target cell. Such delivery is via fusogenic BVs that are released from the FACT cells and that subsequently bind to the target cell. In certain aspects of this embodiment, the cargo molecules are therapeutic molecules. The therapeutic molecules carried by the fusogenic BVs may be, but are not limited to, DNA molecules, RNA molecules, peptides or proteins that are therapeutic for a selected disease or condition. Exemplary peptides and proteins include, but are not limited to, enzymes such as gene-editing enzymes, including base editors, prime editors, TALE nucleases, and CRISPR/Cas,zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

In a fifth embodiment, the invention is directed to methods for delivering one or more cargo molecules to a target cell of a subject, comprising administering to a subject in need thereof a population of FACT cells of the present invention. Upon administration of the FACT cells to the subject, fusogenic BVs are released from the FACT cells and subsequently bind to target cells of the subject. Cargo molecules are then released into the target cells. In certain aspects, cargo molecules encompassed by the fusogenic BVs are DNA molecules, RNA molecules, peptides or proteins. Exemplary peptides and proteins include, but are not limited to, enzymes such as gene-editing enzymes, including base editors, prime editors, TALE nucleases, and CRISPR/Cas,zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

In a sixth embodiment, the invention is directed to methods for treating a disease or condition in a subject, comprising administering to a subject in need thereof a population of FACT cells of the present invention. Upon administration of the FACT cells to the subject, fusogenic BVs are released from the FACT cells and subsequently bind to target cells of the subject. Therapeutic molecules are then released into the target cells. In certain aspects, therapeutic molecules encompassed by the fusogenic BVs are DNA molecules, RNA molecules, peptides or proteins that are therapeutic for the disease or condition. Exemplary peptides and proteins include, but are not limited to, enzymes such as gene-editing enzymes, including base editors, prime editors, TALE nucleases, and CRISPR/Cas, zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

In a seventh embodiment, the invention is directed to methods for delivering fusogenic BVs carrying therapeutic molecules to a target cell, comprising culturing a target cell with a population of fusogenic BVs carrying therapeutic molecules under conditions promoting fusion of fusogenic BVs to the target cell. In certain aspects of this embodiment, therapeutic molecules carried by the fusogenic BVs are DNA molecules, RNA molecules, peptides or proteins that are therapeutic for a selected disease or condition. DNA molecules, RNA molecules, peptides and proteins. Exemplary peptides and proteins include, but are not limited to, enzymes such as gene-editing enzymes, including base editors, prime editors, TALE nucleases, and CRISPR/Cas,zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

In an eighth embodiment, the invention is directed to methods for treating a disease or condition in a subject, comprising administering to a subject in need thereof a population of fusogenic BVs carrying therapeutic molecules, wherein the therapeutic molecules are DNA molecules, RNA molecules, peptides or proteins that are therapeutic for a selected disease or condition. DNA molecules, RNA molecules, peptides and proteins. Exemplary peptides and proteins include, but are not limited to, enzymes such as gene-editing enzymes, including base editors, prime editors, TALE nucleases, and CRISPR/Cas,zinc-finger nucleases. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

In a ninth embodiment, the invention is directed to methods for producing fusogenic BVs comprising culturing FACT cells of the present invention under conditions promoting production and release of fusogenic BVs from the FACT cells.

In each of the embodiments and aspects of the invention, the fusogen or protein with cell penetrating properties may be encoded in a viral genome, in the human genome, or the genome of a non-human mammal. As non-limiting examples, the fusogen or protein with cell penetrating properties may be a viral fusogen, for example VSV-G, mammalian syncytins such as syncytin A (SynA), including human SynA (hSynA), human endogenous retrovirus K (HERV-K) envelope, or functional variants thereof. Such functional variants retain the activity of the protein upon which they are based.

In each of the embodiments and aspects of the invention, the biovesicles (BVs) comprising the fusogens and/or proteins with cell penetrating properties may be fully human BVs. Alternatively, the BVs may comprise one or more elements that are not fully human in origin. For example, the BVs may comprise non-human mammalian fusogens with reduced propensity for immunogenicity in humans, or fusogens that have been humanized, i.e. engineered to be less immunogenic (e.g. mouse SynA) to suit in vivo or chronic dosing.

Thus, in each of the embodiments and aspects of the invention, the fusogenic BVs of the invention may be defined as poorly immunogenic in a subject, such as a human. Thus, the fusogenic BVs of the invention may be fully human fusogenic BVs. Alternatively, the fusogenic BVs of the invention may be described as fusogenic BVs having low or poor immunogenicity. Such low or poor immunogenicity refers to the immunogenicity of the fusogenic BVs when administered to a subject, such as a human.

In each of the embodiments and aspects of the invention, the FACT cell may be a T cell, a monocyte, a megakaryocyte, a neuron, an epithelial cell, such as lung epithelial cells, or a stem cell.

In relevant embodiments and aspects thereof, the subject may be suffering from a disease or condition such as, but not limited to, a chronic neurodegenerative disease, such as amyotrophic lateral sclerosis, Alzheimer's disease, or cancer.

In each of the embodiments and aspects of the invention, the gag-like protein may be encoded in the human genome or the genome of a non-human mammal. As non-limiting examples, the gag-like protein may be a viral gag protein, for example human Arc (hArc) or PEG10, or functional variants thereof.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Transfer of genetically-encoded payload by engineered FACT cell to target cells. (A) Schema of experiment set up using a luciferase complementation assay. FACT cells engineered to express the C-terminal fragment of NanoLuc were transfected with different fusogen expression constructs and then co-cultured with target cells expressing the N-terminal NanoLuc fragment. Payload transfer was determined by luminescence following NanoLuc complementation. (B) Luminescence signal from co-cultures containing genetically engineered 293T-based FACT cells expressing the C-terminal NanoLuc fragment and a fusogen as well SW480 cells expressing the N-terminal NanoLuc fragment.

FIG. 2. Payload transfer by fusogenic biovesicles expressing the HERV-K envelope. Mean fluorescence intensity (MFI) of target 293T cells incubated with concentrated supernatants obtained from 293T cells transiently transfected with payload mCherry as well as an HERV-K expression construct as determined by flow cytometry.

FIG. 3. Effect of fusogen expression on cell manufacturing. (A) mCherry fluorescence (top) and bright-field (bottom) microscopy images of 293T cells transiently transfected with an mCherry payload plasmid and one of the indicated fusogens or a GFP expression plasmid as a negative control at 100-fold magnification. White arrows indicate syncytia. (B) Number of 293T cells 48 h after transfection with the indicated fusogens or a GFP expression plasmid as a negative control as determined by automated cell counter.

FIG. 4. Contribution of gag-like proteins on payload transfer by fusogenic biovesicles. (A) Schema of co-culture experiment using FACT cells transiently transfected with payload, hSYNA, and gag-like protein PEG10 or hArc. mCherry Payload transfer to CellTrace dye Far Red-positive target cells was determined by flow cytometry. (B) Mean fluorescence intensity (MFI) of mCherry in target cells following co-culture with FACT cells transfected with mCherry, hSYNA and the indicated gag-like proteins as determined by flow cytometry.

FIG. 5. Expression of fluorescent reporter proteins co-expressed with fusogen and payload in Jurkat cells engineered using Sleeping Beauty transposase as determined by flow cytometry. The percentage of double positive cells is indicated in each panel.

FIG. 6. Viability of Jurkat cells engineered to express the indicated fusogens as determined by trypan blue staining and automated cell counting.

FIG. 7. Transfer of C-terminal luciferase fragment payload by Jurkat cells engineered to express the indicated fusogens. Engineered Jurkat cells and SW480 cells expressing the N-terminal luciferase fragment were co-cultured at an effector-target ratio of 5:1 for 24 h before addition of furimazine. Luminescence was determine using a Spark multi-mode plate reader (Tecan).

FIG. 8. Transfer of C-terminal luciferase fragment payload using conditioned supernatants collected after 24 h from Jurkat cells engineered to express the indicated fusogens. Conditioned supernatants and SW480 cells expressing the N-terminal luciferase fragment were incubated for 24 h before addition of furimazine. Luminescence was determined using a Spark multi-mode plate reader (Tecan).

FIG. 9. Validation of hSYNA expression in engineered human cells. HSYNA-HA expression as determined by western blot after probing with an anti-HA, anti-TDP-43 or an anti-β-actin (ACTB) antibody in 293T cells transiently transfected with hSYNA-HA expression construct.

FIG. 10. Cell-to-cell delivery of a gene-editing enzyme using hSYNA. (A) Schema of construct used to detect Cre activity in target cells in the absence of Cre (left) and after Cre-mediated excision (right). Black triangles indicate LoxP sites. (B) Conversion of EpCAM+SW480 cells to eGFP following Cre-mediated excision of LoxP cassette after 48 h co-culture with FACT_{Cre} cells in the presence of tamoxifen as determined by flow cytometry. Top panels show all cells, bottom panels show only EpCAM+SW480 cells.

FIG. 11. Delivery of gene-editing enzyme using hSYNA-based fusogenic extracellular vesicles. Conversion of EpCAM+SW480 cells to eGFP following Cre-mediated excision of LoxP cassette after 48 h incubation with supernatants from FACT_{Cre} cells and tamoxifen as determined by flow cytometry.

FIG. 12. Degradation of mutant TDP-43 using FACT cells. 293T cells stably engineered to express wildtype TDP-43 (TDP-43 wt) or mutant TDP-43 (TDP-43C173S/C175S) fused to GFP under control of a tetracycline-inducible promoter were incubated with 293T cells expressing hSYNA alone or together with a TDP-43-specific single-chain variable fragment (3B12A (SEQ ID NO: 7) or Vh7Vk9 (SEQ ID NO: 6)). 24 h after initiating the co-culture, tetracycline was added to the co-culture and after another 24 h, 293T cells were purified by fluorescence activated cell sorting. Cells were lysed in RIPA buffer and analyzed by western blot using antibodies against GFP antibody and ACTB.

FIG. 13. Base editor-mediated correction of mutation using FACT technology. Conversion of EpCAM+SW480-eGFPL202 cells following BE-mediated correction of eGFP mutation after 48 h co-culture with FACTBE as determined by flow cytometry.

FIG. 14. Construct Map. pSBbi-RP-3B12A-hSYNA (FACT open-reading frame; SEQ ID NO: 16)).

FIG. 15. Construct Map. pSBbi-RP-Vh7Vk9-hSYNA (FACT open-reading frame; SEQ ID NO: 17)).

FIG. 16. Construct Map. pTwist-CMV-neo-hSYNA (polynucleotide sequence set forth in SEQ ID NO: 18)).

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

Current approaches for the in vivo delivery of therapeutic molecules, such as RNA and proteins, are mainly limited to the use of lipoparticles to package the therapeutic molecules and achieve cell penetration. However, these approaches only provide minimal control over tissue targeting and relatively short duration of drug action, requiring repeated infusions. Similarly, attempts to utilize endogenous human retroviral fusogens for delivery have so far relied on transient expression systems, likely reflecting current thinking that such delivery vehicles will mainly be produced recombinantly to produce large batches of pre-packaged therapeutics, as an alternative to off-the-shelf EVs.

An attractive alternative to in vitro pre-packaged therapeutics would be to stably engineer cells that serve as a constant source of the genetically encoded therapeutic, thus providing various opportunities for the tissue-specific delivery of the therapeutic and representing a considerable advantage especially in the setting of chronic and degenerative diseases.

Endogenous retroviral proteins have been known to exist in the human genome for decades. However, only recently, it has been suggested that human virus-like particles (HVLPs) produced using the endogenous retroviral protein PEG10 may represent an effective option for drug delivery. The engineering of cells to deliver mRNA using HVLPs has not yet been described.

It has also been shown that the gag-like protein Arc is able to form capsids and shuttles mRNA between neurons as well as human embryonic kidney cells overexpressing Arc [1,2]. However, it is currently not known whether Arc-based HVLPs are pseudotyped via endogenous fusogens present in these cells or whether Arc-based capsids are able to fuse with target cells and deliver their payload without a fusogen. This is important because tropism of retroviruses is to a large extent determined by fusogens and pseudotyping may augment transduction efficiency and increase tissue specificity.

Furthermore, while some information regarding the domain structure of PEG10 and Arc are available, it is unknown which elements of the open-reading frames as well as the untranslated regions required for mRNA packaging are essential for HVLP formation and efficient mRNA packaging.

As reported herein, the first stably engineered and safe fully human cell product for the production of fusogenic biovesicles (BVs) has been achieved.

This cell product forms the basis of the FACT (fusogenic biovesicle-producing adoptive cell therapy) platform described herein for the in vivo production of fusogenic BVs for the delivery of genetically encoded cargo molecules. This platform is highly flexible, permitting the targeting of various cell types, including neurons, immune cells, and epithelial cells, and allowing delivery of a wide range of genetically-encoded cargo molecules. Expression of fusogens with or without expression of gag-like proteins, such as human Arc and PEG10, facilitates the transfer of genetically-encoded cargo molecules carried by FACT cells to target cells for expression therein and, when the cargo molecules are therapeutic molecules, the concomitant treatment of diseases and conditions, including various currently incurable diseases.

The approach reported herein represents a paradigm-shifting technology, potentially allowing the persistent and specific targeting of a multitude of biological processes in some of the most devastating and currently incurable diseases, including but not limited to amyotrophic lateral sclerosis (ALS), cystic fibrosis, and multiple highly prevalent solid cancers.

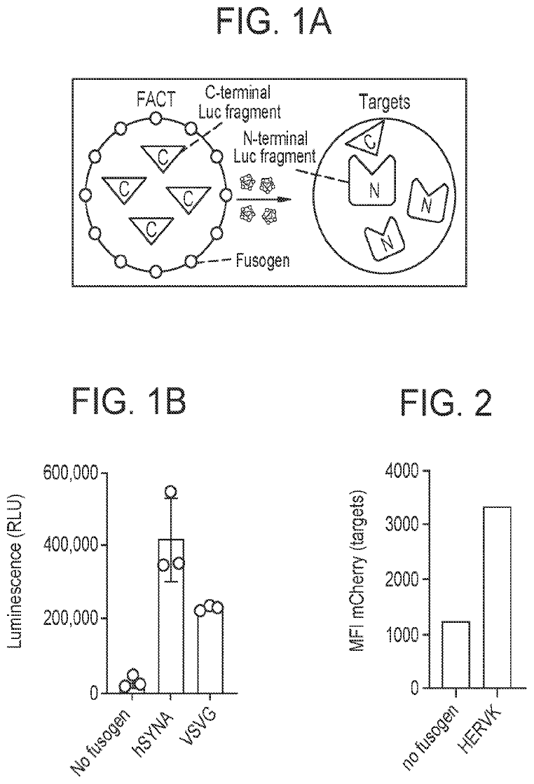

At its core, the FACT approach is made possible by the use of genetically engineered cells expressing fusogenic proteins or peptides that are poorly immunogenic in the subject to which they are administered. When the subject is human, the genetically engineered cells express fully human or humanised or deimmunised fusogenic proteins or peptides, such as those contained in the human genome, that allow the stable production of fully human fusogenic biovesicles or fusogenic BVs having low or poor immunogenicity that can be used to shuttle custom, genetically-encoded therapeutic payloads into neighbouring cells (FIG. 1). Furthermore, as fusogenic proteins are naturally and spontaneously expressed in different healthy human tissues, they may carry limited immunogenicity which may allow long-term persistence of engineered cells expressing these proteins. Suitable proteins and peptides include those from closely-related mammals.

The FACT platform is based on the production of cells (herein “FACT cells”) that produce the elements required for delivery of therapeutic molecules to target cells. As discussed in the paragraphs below, these elements include (i) fusogens or proteins with cell penetrating properties, and in some cases (ii) gag-like proteins. Thus, the FACT cells of the invention encode (i) one or more fusogens or proteins with cell penetrating properties, (ii) one or more genetically encoded cargo molecules, such as therapeutic molecules, and optionally (iii) one or more gag-like proteins.

As used herein, fusogenic proteins or peptides that are “poorly immunogenic” are proteins and peptides that either do not induce an immune response or induce an immune response with undetectable or mild symptoms in a subject that allows cells expressing the fusogenic proteins or peptides to persist in the subject rather than being targeted by the immune system for immediate eradication.

Similarly, as used herein, fusogenic BVs having “low or poor immunogenicity” are fusogenic BVs that either do not induce an immune response or induce an immune response with undetectable or mild symptoms in a subject that allows the fusogenic BVs to persist in the subject rather than being targeted by the immune system for immediate eradication.

Fusogens and Proteins with Cell Penetrating Properties

Fusogens, also known as fusion proteins, are proteins that allow membranes to fuse by overcoming the repulsion between membranes. Viruses use fusogens to efficiently merge with their target cells and deliver their payload. Viruses encode their own fusogens, such as the vesicular stomatitis virus (VSV) G fusion protein, which has broad tropism and is widely used in the production of recombinant viruses for therapeutic applications. However, viral envelope proteins including fusogens that are expressed on the surface of host cells prior to viral budding may serve as a key target of antibody-mediated immunity. Therefore, careful thought is required in selecting suitable fusogens for use in the embodiments of the present invention.

Fusogens that may be used in the FACT cells of the present invention include endogenous human or other mammalian retroviral fusogens, such as human endogenous retrovirus K (HERV-K), syncytin A (SynA) (e.g. human SynA), baboon retroviral envelope glycoprotein (BaEV), and the endogenous retroviral envelope protein EnVP(b)1. These proteins are likely to exhibit reduced propensity for immunogenicity [26]. Commonly used non-human or viral fusogens such as fusogens from vesicular stomatitis virus (VSV) may be used as a tool for optimizing different components of FACT system or as a control. In addition, the fusogens from one species may be used in the preparation of BVs from another species. Thus, in terms of fusogens to be used in human applications for example, suitable fusogens may be non-human fusogens that have been humanized or deimmunised, i.e. engineered to be less immunogenic in a human. Suitable fusogens include those from closely-related mammals.

In addition to these specific fusogens, it should be understood that other proteins and peptides having cell penetrating properties may be used in the embodiments of the invention, either in place of traditional fusogens, such as those mentioned above, or in addition to these fusogens. Suitable cell-penetrating proteins and peptides encompassed within the scope of the invention include, but are not limited to:

| | RALA |
|---|---|
| SEQ ID NO: 8 | |
| | (WEARLARALARALARHLARALARALRACEA;); |
| | PF14 |
| SEQ ID NO: 9 | |
| | (Stearyl-AGYLLGKLLXXLAAAALXXLL, where X is |
| | ornithine;); |
| (SEQ ID NO: 10) | |
| | PFVYLI; |
| | KALA |
| SEQ ID NO: 11 | |
| | (WEAKLAKALAKALAKHLAKALAKALKA;); |
| | GALA |
| SEQ ID NO: 12 | |
| | (WEAALAEALAEALAEHLAEALAEALEALAA;); |
| | LAH4-L1 |
| SEQ ID NO: 13 | |
| | (KKALLAHALHLLALLALHLAHALKKA;); |
| | KL4 |
| SEQ ID NO: 14 | |
| | (KLLLLKLLLLKLLLLKLLLLK;); |
| | and |
| | OligoArgAib |
| SEQ ID NO: 15 | |
| | (RRXRRXRRXRRXRRX, where X is a-aminoisobutyric |
| | acid (Aib);.) |

In addition to proteins and peptides having the innate ability to penetrate cells, proteins can be engineered to have cell penetrating properties. Such engineered proteins are encompassed within the scope of the invention. For example, cell-permeable proteins may be engineered by genetically grafting a short cell-penetrating peptide (CPP) to an exposed loop of a protein of interest.

Functional variants of the fusogens and proteins with cell penetrating properties defined above may also be used in the various aspects and embodiments of the invention. These functional variants will retain the activity of the protein upon which they are based, but have at least 50%, 55%, 60%, 65%, 70%, 75%, 80%, 85%, 90%, 91%, 92%, 93%, 94%, 95%, 96%, 97%, 98% or 99% sequence identity with the protein upon which they are based.

The FACT cells, independently of the gag-like proteins, can be engineered to either transiently or stably express the fusogens. For transient expression, actual mRNA or recombinant proteins, or any plasmids containing promoters active in eukaryotic cells, such as CMV, EF1A, PGK, and SFFV may be used. For stable expression, transposon-based plasmids together with a transposase, or adeno/retroviral transfer plasmids using the same type of promoters or promoters contained within the long-terminal repeats of the viral vectors may be used. Suitable means for preparing FACT cells expressing the proteins include, but are not limited to, calcium phosphate, lipofection, nucleofection, electroporation, adeno-/retroviral transduction.

The same plasmid/vector can encode both the gag proteins and the fusogens, when gag proteins are present.

As suggested above, the fusogens and proteins having cell penetrating properties can be genomic (chromosomal) or non-genomic sequences. Non-genomic sequences are extra-chromosomal sequences such as those of a plasmid or virus present in the FACT cells.

### Fusogenic BVs

The present invention also encompassed fusogenic BVs comprising (i) one or more fusogens or proteins with cell penetrating properties, (ii) one or more cargo molecules, and (iii) optionally one or more gag-like proteins. It should be understood that the fusogenic BVs themselves are constructed of (i) the fusogens or proteins with cell penetrating properties, and (ii) the gag-like proteins, when present, to form a structure. The cargo molecules are present inside of the structure and thus carried within the fusogenic BVs.

As suggested above, in each of the embodiments and aspects of the invention, the fusogenic BVs may be fully human fusogenic BVs. Alternatively, the BVs may comprise one or more elements that are not fully human in origin. For example, the BVs may comprise non-human mammalian fusogens with reduced propensity for immunogenicity in humans, or fusogens that have been humanized, i.e. engineered to be less immunogenic (e.g. mouse SynA) to suit in vivo or chronic dosing.

Thus, in each of the embodiments and aspects of the invention, the fusogenic BVs of the invention may be described as fully human fusogenic BVs. Alternatively, the fusogenic BVs of the invention may be described as fusogenic BVs having low or poor immunogenicity. Such low or poor immunogenicity refers to the immunogenicity of the fusogenic BVs when administered to a subject, such as a human.

As used herein, the term “fully human” refers to peptides and proteins that are encoded by the human (Homo sapiens) genome (e.g. human SynA), and biovesicles that only comprise and/or contain peptides, proteins and other molecules that are encoded by the human genome, derived from the human body, or produced by the human body.

### Cargo Molecules

As indicated above, the fusogenic biovesicles produced by the FACT cells of the invention shuttle cargo molecules to target (receiving) cells. The cargo molecules will typically be therapeutic molecules. However, the cargo molecules may also have other functions, such as a signalling function if the cargo molecule is used in the context of a cell-based assay system.

The FACT cells can be engineered to either transiently or stably express the genetically encoded cargo molecules, such as DNA molecules, RNA molecules, peptides or proteins. Exemplary peptides and proteins include, but are not limited to, enzymes such as gene-editing enzymes, including base editors, prime editors, TALE nucleases, and CRISPR/Cas,zinc-finger nucleases. Base editors (BE) are engineered proteins that are able to alter individual nucleotides at defined positions in a cell's genome without introducing double-strand breaks. BEs include, but are not limited to, cytosine and adenine base editors. Additional exemplary DNA molecules, RNA molecules, peptides and proteins are those reducing RNA and protein levels in target cells, including, but not limited to, small-interfering RNA, short-hairpin RNA, and proteins and peptides containing domains targeting molecules for proteasomal degradation. Additional exemplary DNA molecules, RNA molecules, peptides and proteins include those correcting aberrant protein function, for example by altering protein conformation or trafficking, or providing wildtype protein function.

For transient expression, DNA, RNA, recombinant peptides or recombinant proteins, or any plasmids containing promoters active in eukaryotic cells, such as CMV, EF1A, PGK, SFFV, U6 may be used. For stable expression, transposon-based plasmids together with a transposase, or adeno/retroviral transfer plasmids using the same type of promoters or promoters contained within the long-terminal repeats of the viral vectors may be used. A suitable plasmid for both transient and stable expression of cargo molecules is, for example, pSBBi-RP, a Sleeping Beauty plasmid.

Suitable means for preparing FACT cells expressing the cargo molecules include calcium phosphate, lipofection, nucleofection, electroporation, adeno-/retroviral transduction.

As should be apparent, the identity of the cargo molecules that makes up the payload can vary widely and is not limited to DNA, RNA, peptides or specific proteins themselves.

### Gag-Like Proteins

The gag-like proteins that may be expressed by the FACT cells of the invention are retroviral gag-like proteins that are endogenous proteins encoded by the human genome or genome of other mammals.

As mentioned above, PEG10 is a suitable gag-like protein for use in the FACT cells of the invention. PEG10 contains not only a gag-like sequence consisting of a capsid and nucleocapsid polypeptide but also a partial pol segment, likely encoding a protease (PR) and reverse transcriptase (RT). To reduce the size of the DNA elements required for FACT cell production, PR and RT domains may be deleted from the PEG10 ORF. It has been shown that the RT domain is not required for efficient cargo transport.

The human Arc gene also encodes a gag-like capsid protein that may be used to increase cargo transfer by fusogenic BVs.

Other gag-like proteins that can be expressed by FACT cells include, but are not limited to, Pnma1, Pnma3, Pnma5, Pnma6a, Pnma8, Asprv1, Moap1, Zcchc12, and Rtl1.

Functional variants of the gag-like proteins defined above may also be used in the various aspects and embodiments of the invention. These functional variants will retain the activity of the protein upon which they are based, but have at least 50%, 55%, 60%, 65%, 70%, 75%, 80%, 85%, 90%, 91%, 92%, 93%, 94%, 95%, 96%, 97%, 98% or 99% sequence identity with the protein upon which they are based.

The FACT cells can be engineered to either transiently or stably express the gag-like proteins. For transient expression, actual mRNA or recombinant proteins, or any plasmids containing promoters active in eukaryotic cells, such as CMV, EF1A, PGK, SFFV may be used. For stable expression, transposon-based plasmids together with a transposase, or adeno/retroviral transfer plasmids using the same type of promoters or promoters contained within the long-terminal repeats of the viral vectors may be used.

Suitable means for preparing FACT cells expressing the proteins include calcium phosphate, lipofection, nucleofection, electroporation, adeno-/retroviral transduction.

### Engineered Cell Types

The FACT cells of the invention may use any cell type that will produce fusogenic biovesicles that can carry selected cargo molecules, such as proteins or RNA.

Suitable cell types include, but are not limited to, cells of the immune system, such as T cells, natural killer cells and monocytes; megakaryocytes; neurons; epithelial cells, such as lung epithelial cells; and stem cells. An exemplary cell type is T cells.

### Diseases

As indicated above, the FACT cells of the invention can be used, inter alia, in the treatment of certain diseases and conditions. Exemplary diseases include, but are not limited to, chronic neurodegenerative diseases, such as amyotrophic lateral sclerosis and Alzheimer's disease, cancer, mitochondrial diseases, lysosomal deficiencies, and genetic diseases resulting from loss of heterozygosity.

### Formulations

Depending on the manner in which the FACT cells are being used, a population of FACT cells can be formulated for administration to a subject, such as a human subject, as a pharmaceutical composition. These pharmaceutical compositions will comprise one or more populations of FACT cells and a suitable carrier or excipient.

The fusogenic BVs of the invention can also be formulated for administration to a subject, such as a human subject, as a pharmaceutical composition. These pharmaceutical compositions will comprise one or more populations of fusogenic BVs and a suitable carrier or excipient.

The pharmaceutical compositions of the present invention may be formulated, for example, for oral, sublingual, intranasal, intraocular, rectal, transdermal, mucosal, pulmonary, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of pharmaceutical compositions can be used to effect such administration.

Depending on the means of administration, the dosage may be administered all at once, such as with an oral formulation in a capsule or liquid, or slowly over a period of time, such as with an intramuscular or intravenous administration.

Administration frequencies for the pharmaceutical compositions of the present invention include 4, 3, 2 or once daily, every other day, every third day, every fourth day, every fifth day, every sixth day, once weekly, every eight days, every nine days, every ten days, bi-weekly, monthly, and bi-monthly. The duration of treatment will be based on the condition being treated and will be best determined by the attending physician.

### Methods

As suggested above, the present invention includes methods for utilizing the fusogenic BVs and FACT cells defined herein.

For example, the invention includes methods for delivering one or more cargo molecules to a target cell, comprising culturing a target cell with a fusogenic BV of the present invention under conditions promoting delivery of a cargo molecule from the fusogenic BV to the target cell. Such delivery is via binding of the fusogenic BVs to the target cell and release of the cargo molecules into the target cell.

The invention also includes methods for delivering one or more cargo molecules to a target cell, comprising culturing a target cell with a FACT cell of the present invention under conditions promoting delivery of a cargo molecule from the FACT cell to the target cell. Such delivery is via fusogenic BVs that are released from the FACT cells and that subsequently bind to the target cell.

The invention further includes methods for delivering one or more cargo molecules to a target cell of a subject, comprising administering to a subject in need thereof a population of FACT cells of the present invention. Upon administration of the FACT cells to the subject, fusogenic BVs are released from the FACT cells and subsequently bind to target cells of the subject. Cargo molecules are then released into the target cells.

In addition, the invention includes methods for treating a disease or condition in a subject, comprising administering to a subject in need thereof a population of FACT cells of the present invention. Upon administration of the FACT cells to the subject, fusogenic BVs are released from the FACT cells and subsequently bind to target cells of the subject. Therapeutic molecules are then released into the target cells.

Moreover, the invention includes methods for delivering fusogenic BVs carrying therapeutic molecules to a target cell, comprising culturing a target cell with a population of fusogenic BVs carrying therapeutic molecules under conditions promoting fusion of fusogenic BVs to the target cell.

The invention also includes methods for treating a disease or condition in a subject, comprising administering to a subject in need thereof a population of fusogenic BVs carrying therapeutic molecules, wherein the therapeutic molecules are RNA molecules, peptides or proteins that are therapeutic for a selected disease or condition.

The invention further includes methods for producing fusogenic BVs comprising culturing FACT cells of the present invention under conditions promoting production and release of fusogenic BVs from the FACT cells.

In the relevant embodiments and aspects provided above, the cargo molecules may be therapeutic molecules. The cargo molecules may be, but are not limited to, RNA molecules, peptides or proteins as defined above. The therapeutic molecules may be, but are not limited to, RNA molecules, peptides or proteins as defined above that are therapeutic for a selected disease or condition

In each of the embodiments and aspects of the invention, the subject is any vertebrate animal including, but not limited to, human, non-human primate, bird, horse, cow, goat, sheep, a companion animal, such as a dog, cat or rodent, or other mammal.

### III. Examples

### Example 1: Cells Producing Fully Human BVs are an Effective Approach for the Intercellular Transfer of Genetically-Encoded Cargo

Persistent delivery of genetically-encoded therapeutics using cells engineered to produce fusogenic biovesicles comprising endogenous human fusogens may be an effective approach to treating disease. To this end, a new assay was developed to measure the cytosolic delivery of a genetically-encoded payload using a luciferase complementation system (FIG. 1A). Specifically, a luciferase-complementation assay was developed using a previously described split NanoLuc pair [32], with the C-terminal luciferase fragment stably expressed in the FACT cells and the N-terminal fragment stably expressed in the target cells. Upon transfer of the C-terminal fragment from the FACT cells co-transfected with a fusogen, both fragments readily assemble in the cytosol of the target cell, causing luminescence upon incubation with the NanoLuc substrate furimazine.

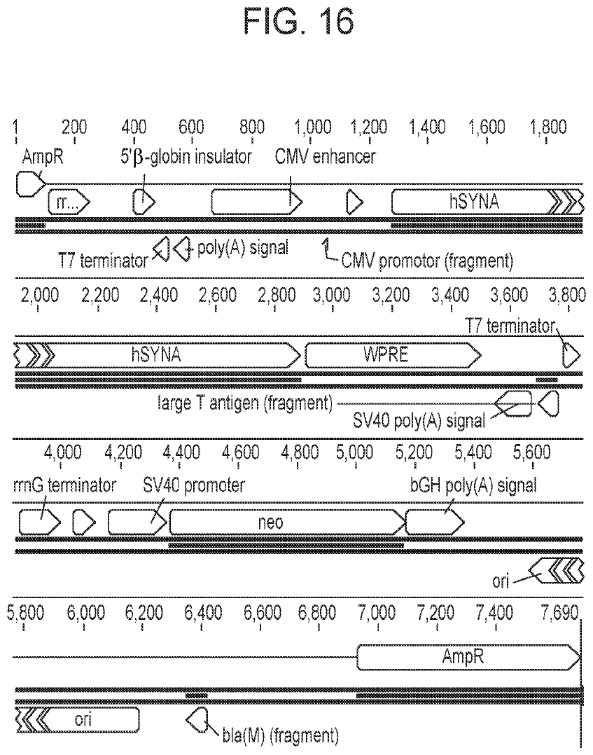

Specifically, 293T-based FACT cells expressing the C-terminal NanoLuc fragment (cLuc) were engineered by co-transfection of a plasmid containing inverted tandem repeat (ITR)-flanked C-terminal NanoLuc within the previously described pSBbi backbone together with a plasmid encoding the optimized Sleeping Beauty SB100X using Lipofectamine 2000. At the same time, an N-terminal NanoLuc (nLuc) overexpressing colorectal cancer cell line SW480 was generated via lentiviral transduction using the LeGO system followed by fluorescence-activated cell sorting on a FACSaria cell sorter (BD). To determine the effect of fusogen overexpression on payload transfer and subsequent Nano-Luc complementation, 5×10⁵ FACT cells were transiently transfected with the human fusogen syncytin A (hSYNA, SEQ ID NO: 1) using the construct shown in FIG. 16 or the established fusogen vesicular stomatitis virus G (VSV-G) envelope (control) in a 24-well plate following the manufacturer's instructions. After 24 h, FACT and target cells were washed twice with 1 ml PBS and cLuc-expressing FACT cells transfected with fusogen constructs were subsequently co-cultured with nLuc-expressing SW480 cells at an effector-target ratio of 5:1. After 24 h, cells were transferred to a black non-binding plate (Greiner) and furimazine substrate (Promega) was added according to the manufacturer's instructions. Luminescence signal was read on a Spark multi-mode plate reader (Tecan) after a 5 min incubation at 37° C.

It was found that expression of the human protein hSYNA and VSV-G control both substantially increased luminescence signal in the target cells (FIG. 1B), confirming efficient transfer of a genetically encoded payload from a FACT cell to a target cell. This result indicated that cells engineered to produce fully human fusogenic biovesicles are an effective approach to persistently transfer payloads to relevant target cells.

### Example 2: Fully Human Fusogenic Biovesicles can be Generated Using the HERV-K Envelope

In the previous example, it was demonstrated that the human protein syncytin A can be used to engineer cells producing fully human fusogenic biovesicles. To determine whether there are alternative endogenous fully human fusogens that could be used, a literature search was performed for potential envelope proteins of endogenous human retroviruses. The envelope protein of the human endogenous retrovirus K (HERV-K) was identified as a possibility. HERV-K is the most recently identified endogenized human retrovirus and its envelope protein has previously been suggested to have potentially broad tropism [37]. It was therefore hypothesized that it may represent a promising alternative to syncytins in the context of the engineering of fusogenic biovesicles.

FACT cells were generated expressing mCherry as the payload and HERV-K (SEQ ID NO 2) as the fusogen. Specifically, 70-80% confluent 293T cells were transiently transfected in T75 flasks with an mCherry expression plasmid and an HERV-K expression plasmid, both in the pcDNA3.4 backbone, using Lipofectamine 2000. After 24 h, the 293T cells were washed with PBS and fresh complete culture medium was added. After another 24 h, the supernatants were harvested and filtered using a 0.45 μm polyethersulfone filter (MilliporeSigma) and the filtered supernatants were concentrated 10-fold using commercially available Retro-X concentrator solution (Takara). Next, wells of a 24-well tissue culture plate were coated with 10 μg/ml retronectin (Takara), the wells were blocked with 2% bovine serum albumin (BSA), and concentrated supernatants were immobilized on retronectin-coated plates by centrifugation according to the manufacturer's instructions. After removing supernatants, 5×10⁵ untransfected 293T cells were added to wells coated with fusogenic vesicles and incubated for 24 h at 37° C./5% CO₂. After 24 h, cells were harvested by trypsinization, filtered through a 70 μm cell strainer (BD), and analyzed on an LSR II flow cytometer (BD).

It was found that use of the HERV-K envelope proteins substantially increased transfer of the mCherry payload to target cells (FIG. 2), demonstrating that the HERV-K envelope represents an alternative to syncytins for the generation of fusogenic vesicles as a standalone drug or when delivered by engineered FACT cells.

### Example 3: Biovesicles Using Human Fusogens can be Manufactured Efficiently

Fusogenic proteins are able to overcome the repulsion between plasma cell membranes and actively facilitate the fusion of multiple cells or a cell with a fusogenic biovesicle. The functionality of FACT cells relies on the expression of individual fusogenic proteins and it was hypothesized that expression of some fusogens may cause fusion not only by the fusogenic vesicles to target cells but also between the engineered FACT cells and can lead to reduced FACT cell viability and production efficiency.

To investigate, 293T cells were plated at 5×10⁵ cells/well in a 24-well plate and transiently transfected with expression constructs for hSYNA, mouse syncytin A (mSYNA, SEQ ID NO: 3), HERV-K, VSV-G (control) or GFP (green fluorescent protein) as a negative control together with an mCherry payload plasmid using Lipofectamine 2000 according to the manufacturer's instructions. After 24 h, the cells were analyzed by fluorescence and bright field microscopy, and the remaining cells were counted following trypsinization using an automated cell counter (ThermoFisher).

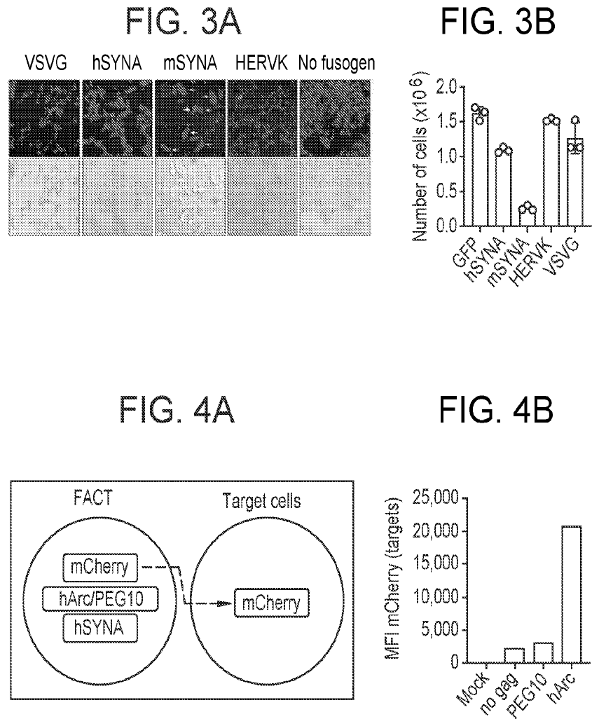

It was found that expression of mSYNA caused the formation of fused cell conglomerates or syncytia, while this was not the case for hSYNA, HERV-K, or VSV-G (FIG. 3). Accordingly, dramatically reduced cell yields were observed in cells transfected with mSYNA. Also, slightly reduced cell numbers were observed after transfection with hSYNA and VSV-G (control). In contrast, transfection with the HERV-K envelope did not change yields compare to GFP-transfected cells. These data demonstrate that the production and long-term persistence of cells transiently or stably expressing mSYNA may be limited and that other fusogens, such as hSYNA and HERV-K, may represent better choices in this setting.

### Example 4: Addition of Gag-Like Proteins to Fusogenic Biovesicles can Enhance Cargo Transfer

It was found that fusogenic biovesicles using only human proteins is an efficient minimal approach for the delivery of payloads to target cells either as a standalone approach or when delivered by engineered FACT cells.

While fusogenic proteins are sufficient for the transfer of payloads, it may be possible to increase the transfer efficiency of fusogenic biovesicles in some cases. Conventional retroviruses use gag proteins to specifically load and encapsulate their viral payloads. Therefore, experiments were also conducted to analyze whether addition of fully human gag-like proteins, such as Paternally Expressed-10 (PEG10) and Activity Regulated Cytoskeleton Associated Protein (hArc) encoded in the human genome are able to further increase payload transfer efficiency using the human fusogen hSYNA.

In the case of PEG10, the reverse transcriptase domain was deleted from its open-reading frame (SEQ ID NO: 4). A direct co-culture experiment (FIG. 4) was performed by first plating 5×10⁵ 293T cells per well in a 24-well plate and subsequently transfecting those cells with an mCherry payload construct, together with expression constructs for hSYNA (using the construct shown in FIG. 16) and PEG10 (SEQ ID NO: 1) or hArc (SEQ ID NO: 5) using Lipofectamine 2000 according to the manufacturer's instructions. After 24 h, FACT cells were washed twice with 1 ml PBS and untransfected 293T target cells were stained with CellTrace dye Far Red (ThermoFisher). FACT cells were co-cultured with 5×10⁵ target cells at an effector-target ratio of 5:1 for 24 h at 37° C./5% CO₂. After 24 h, cells were harvested by trypsinization, filtered through a 70 μm cell strainer (BD), and analyzed on an LSR II flow cytometer (BD). Target cells were identified by CellTrace dye Far red positivity.

It was again found in this modified direct co-culture setting, that use of only a fusogen is sufficient to transfer payloads to target cells (FIG. 4). Addition of both PEG10 and hArc increased this payload transfer. These data demonstrate that depending on requirements and context it may be beneficial to provide gag-like proteins when generating FACT cells or producing fully human fusogenic particles.

### Example 5: T Cell-Mediated Payload Transfer

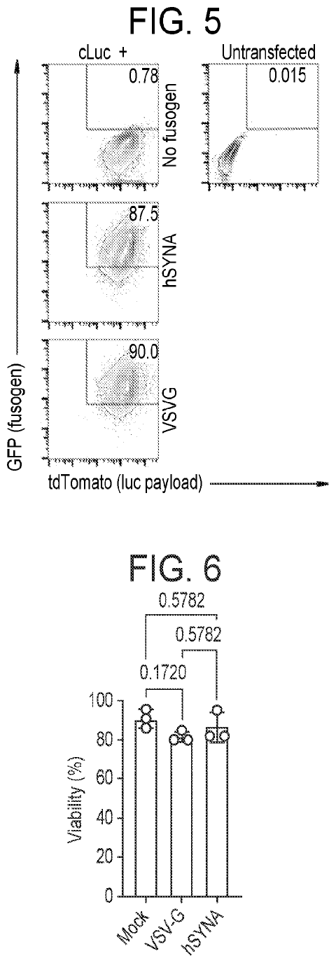

T cells can provide an ideal vehicle for the delivery of therapeutic payloads, due to their long-term persistence and demonstrated clinical safety. However, prior to the present invention, it was unknown whether T cells can be engineered to overexpress fusogenic biovesicles (BVs) that can transfer payloads to relevant target cells. The potential impact of fusogen expression on T cell viability was also unknown. We therefore engineered the human T cell line Jurkat using transposase-based gene transfer to express the C-terminal NanoLuc fragment together with human Arc and the fusogens VSV-G or human syncytin A (using the construct shown in FIG. 16). We show that Jurkat cells expressing fusogens can be engineered efficiently (FIG. 5) and that fusogen expression does not affect T cell viability (FIG. 6).

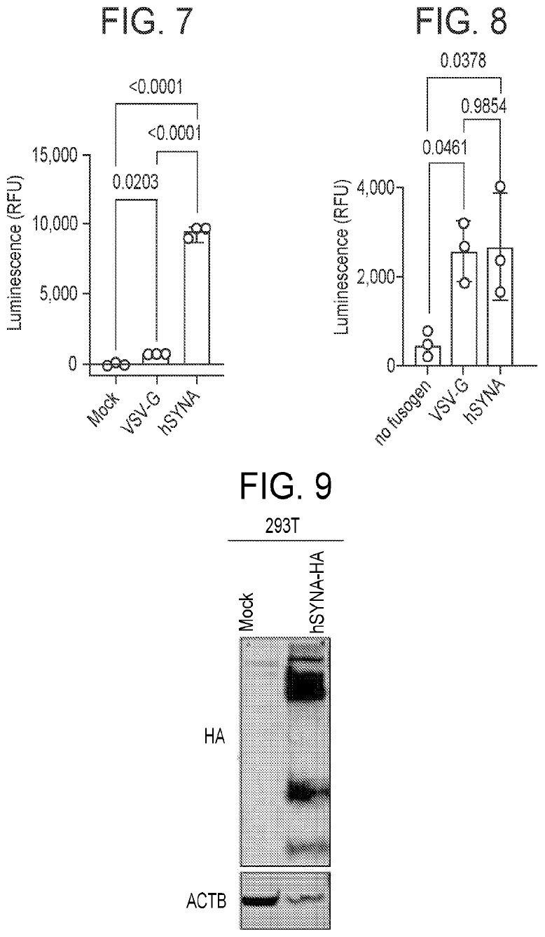

We next co-cultured colorectal SW480 cells expressing the N-terminal luciferase fragment with the engineered Jurkat cells and determined payload transfer by luminescence assay. We observed significant levels of payload transfer using both human syncytin A and VSV-G compared to T cells only expressing the payload construct (FIG. 7). In addition, we show that significant luciferase payload transfer can also be achieved using supernatants collected from T cells engineered to express the luciferase payload and a fusogen (FIG. 8). These data show that T cells can be engineered efficiently using the methods described herein to produce fusogenic BVs and transfer payloads to a target cell.

### Example 6: Validation of hSYNA Expression in Engineered Human Cells

Payload transfer relies on the overexpression of hSYNA in human FACT cells. Therefore, an experiment was conducted to determine if engineered FACT cells indeed show relevant expression of hSYNA following stable integration of an hSYNA expression cassette using Sleeping Beauty. HSYNA remains a relatively little studied protein and few reagents exist to detect its expression. Therefore, the full open-reading frame of hSYNA fused to a C-terminal hemagglutinin (HA) tag was synthesized and cloned into the mammalian expression plasmid pcDNA3.4. 293T cells were transfected with this construct using lipofection to transiently express hSYNA-HA. (FIG. 9). Lysates were generated using RIPA buffer and subjected to SDS-PAGE. Proteins were transferred to a nitrocellulose membrane using an iBlot 2 device. The membrane was probed with an anti-HA and a β-actin (ACTB) antibody and protein levels were visualized by chemiluminescence. In the transfected cells, high levels of hSYNA-HA were detected (FIG. 9).

### Example 7: Cell-to-Cell Delivery of a Gene-Editing Enzyme Using hSYNA

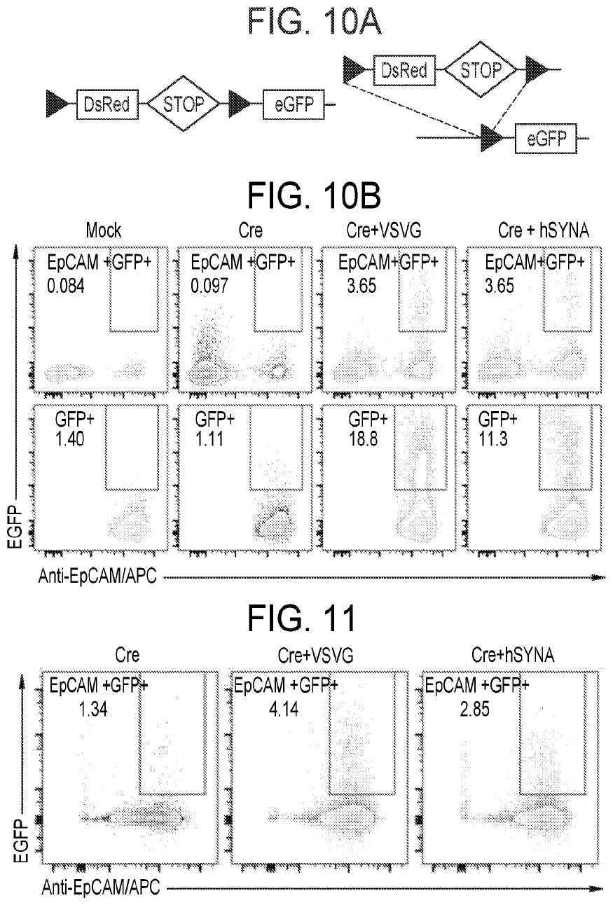

The efficient delivery of gene-editing enzymes is a promising approach for the treatment of human disease, e.g. by correcting hereditary or acquired genetic defects. To determine if FACT cells based on hSYNA are able to deliver such gene-editing enzymes, a Cre-LoxP based reporter system was created (FIG. 10A). Specifically, colorectal EpCAM-positive SW480 cells were engineered using lentivirus to carry a LoxP-flanked DsRed fluorescent reporter including a stop codon followed by an eGFP reporter (SW480-LoxP-DsRed-eGFP) using the pLV-CMV-LoxP-DsRed-LoxP-eGFP plasmid. Following Cre-mediated excision of the LoxP-flanked cassette, SW480 cells switch from DsRed to eGFP. FACT cells were engineered by lipofection to express hSYNA as well as tamoxifen-inducible Cre using the pcDNA3.1-CMV-CFP;UBC-Cre25nt plasmid. FACT_{Cre} cells were co-cultured with SW480-LoxP-DsRed-eGFP cells in the presence of 3 μM tamoxifen for 48 h at 37° C. Co-cultured cells were stained with an APC-conjugated anti-EpCAM antibody (clone: 9C4) and analyzed by flow cytometry. It was found that FACT_{Cre} cells efficiently converted SW480-LoxP-DsRed-eGFP cells to eGFP (FIG. 10B), with the top panel showing all cells and the bottom panel only showing the EpCAM-positive target cells. These data demonstrate that the approach disclosed herein is effective at delivering a gene-editing enzyme to target cells.

### Example 8: Generation of Fusogenic Extracellular Vesicles Capable of Delivering of Gene-Editing Enzymes

Extracellular vesicles (EV) are a promising tool for the delivery of therapeutic payloads, especially gene-editing enzymes, due to their high biocompatibility and low immunogenicity. The most commonly used protein to generate fusogenic EVs is the vesicular stomatitis virus G (VSVG) envelope protein. While VSVG is a highly efficient fusogen with broad tropism, as a large viral protein it elicits potent immune responses limiting efficacy and potentially preventing repeat infusions.

FACT cells are able to efficiently deliver gene-editing enzymes when co-cultured directly with target cells. In addition, FACT cells could serve as the source of fusogenic EVs, an alternative method of delivering therapeutic payloads. Therefore, supernatants were collected from FACT_{Cre} cells engineered using different fusogens. Supernatants were added to SW480-LoxP-DsRed-eGFP cells and incubated for 48 h in the presence of tamoxifen. Conversion of the cells to eGFP was determined by flow cytometry as described above. It was found that supernatants harvested from FACT_{Cre} cells were able to induce eGFP expression in SW480 cells, indicating that FACT cells generate fusogenic EVs (FIG. 11).

### Example 9: Delivery of Targeted Protein Degraders Using FACT Cells

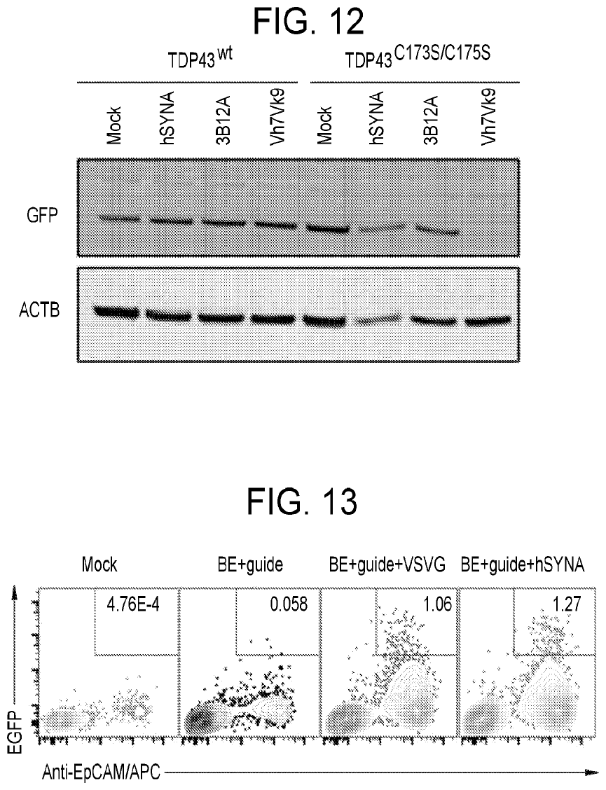

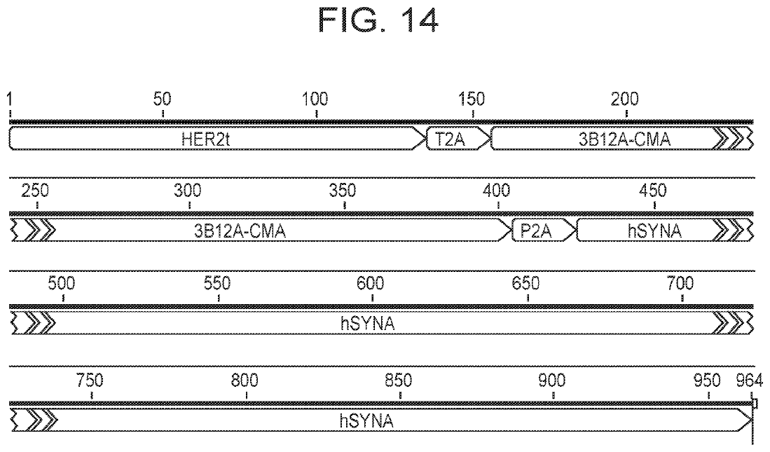

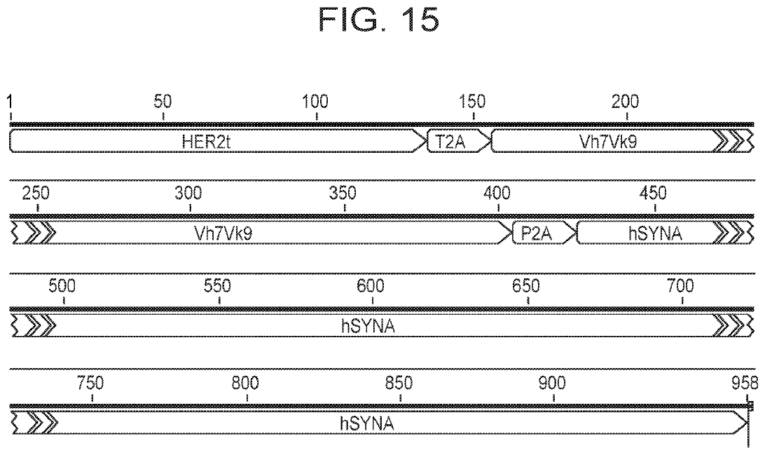

Targeted degradation of pathogenic proteins is another potential application of FACT cells. Therefore, the ability of FACT cells to degrade TDP-43, a key protein found in neurofibrils in patients with amyotrophic lateral sclerosis (ALS), was determined. Mutant TDP-43C173S/C175S shows rapid aggregation when expressed in human cells. Mutant and wildtype TDP-43 were cloned into the tetracycline-inducible pLVX-TetOne plasmid using restriction cloning. 293T cells were engineered by lentiviral transduction to express either wildtype TDP-43 or TDP-43C173S/C175S fused to GFP under control of a tetracycline-inducible promoter. 293T cells were engineered to stably express hSYNA and one of two single-chain variable fragments (clones 3B12A and Vh7Vk9) targeting aggregated TDP-43 [21, 24] (see FIGS. 14 and 15). FACT cells and TDP-43-GFP-expressing target cells were co-cultured for 24 h before addition of tetracycline. After 24 h, target 293T cells were purified by fluorescence activated cell sorting. Cells were lysed in RIPA buffer and analyzed by western blot using antibodies against GFP antibody and ACTB. FACT cells showed efficient reduction in total TDP-43 in target cells as determined by western blot using FACT cells engineered to deliver the TDP-43-specific scFv Vh7Vk9 (SEQ ID NO: 6) (FIG. 12). These data indicate that FACT cells are able to cause the degradation of specific proteins in target cells.

### Example 10: FACT-Mediated Delivery of a Genome Editor

In vivo gene editing using genome editors such as CRISPR-Cas9, Base Editors, and Prime Editors have great therapeutic potential. Base editors (BE) are engineered proteins that are able to alter individual nucleotides at defined positions in a cell's genome without introducing double-strand breaks. BEs are currently being explored in clinical trials for the treatment of various diseases [38] but their inefficient delivery using current approaches limits their therapeutic applications. To test whether FACT can serve as a technology for the delivery of BEs, SW480 target cells expressing mutated eGFPL202 were generated by lentiviral transduction using the plenti-CMV-mCherry-T2A-GFPL202 plasmid. Excitation of eGFPL202 does not result in characteristic eGFP fluorescence [39]. FACT cells were engineered by lipofection to express hSYNA, an adenine BE using the A3Ai-Cas9n-UGI-NLS construct, as well as a guide RNA targeting the L202 mutation in eGFP and co-cultured target cells with these cells for 48h. Increased GFP expression in SW480-eGFPL202 target cells was observed after co-culture with FACT cells by flow cytometry after staining with an anti-EpCAM antibody as described above (FIG. 13). This finding demonstrates that FACT technology is able to deliver functional BEs together with guide RNA to correct mutations on the DNA level, an application with high therapeutic potential.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Bhat, B., Karve, S. & Anderson, D. G. mRNA therapeutics: beyond vaccine applications. Trends Mol. Med. 27, 923-924 (2021).
2. Melenhorst, J. J. et al. Decade-long leukaemia remissions with persistence of CD4 CAR T cells. Nature 602, 503-509 (2022).
3. Pastuzyn, E. D. et al. The Neuronal Gene Arc Encodes a Repurposed Retrotransposon Gag Protein that Mediates Intercellular RNA Transfer. Cell 172, 275-288.e18 (2018).
4 Ashley, J. et al. Retrovirus-like Gag Protein Arc1 Binds RNA and Traffics across Synaptic Boutons. Cell 172, 262-274.e11 (2018).
5. Segel, M. et al. Mammalian retrovirus-like protein PEG10 packages its own mRNA and can be pseudotyped for mRNA delivery. Science 373, 882-889 (2021).
6. Counsell J R, De Brabandere G, Karda R, Moore M, Greco A, Bray A, et al. Re-structuring lentiviral vectors to express genomic RNA via cap-dependent translation. Mol Ther Methods Clin Dev 2021 Mar. 12; 20, 357-365.
7. Mock U, Riecken K, Berdien B, Qasim W, Chan E, Cathomen T, et al. Novel lentiviral vectors with mutated reverse transcriptase for mRNA delivery of TALE nucleases. Sci Rep 2014 Sep. 18; 4, 6409.
8. Magnani, C. F. et al. Sleeping Beauty-engineered CAR T cells achieve antileukemic activity without severe toxicities. J. Clin. Invest. 130, 6021-6033 (2020).
9. Konermann, S. et al. Genome-scale transcriptional activation by an engineered CRISPR-Cas9 complex. Nature 517, 583-588 (2015).
10. Xu, X. et al. Engineered miniature CRISPR-Cas system for mammalian genome regulation and editing. Mol. Cell 81, 4333-4345.e4 (2021).
11. Charlesworth, C. T. et al. Identification of preexisting adaptive immunity to Cas9 proteins in humans. Nat. Med. 25, 249-254 (2019).
12. Ivics, Z. et al. Molecular reconstruction of Sleeping Beauty, a Tcl-like transposon from fish, and its transposition in human cells. Cell 91 (4), 501-10 (1997).
13. Jin, Z. et al. The hyperactive Sleeping Beauty transposase SB100X improves the genetic modification of T cells to express a chimeric antigen receptor. Gene Ther 18 (9), 849-56 (2011).
14. Izsvak, Z. et al. Sleeping Beauty, a wide host-range transposon vector for genetic transformation in vertebrates. J Mol Biol 302 (1), 93-102 (2000).
15. Kumar, M. et al. Systematic determination of the packaging limit of lentiviral vectors. Hum Gene Ther 12 (15), 1893-905 (2001).
16. Regulations.gov. https://www.regulations.gov/docket/FDA-2018-N-0410.
17. Biogen and Ionis Announce Topline Phase 1 Study Results of Investigational Drug in C9orf72 Amyotrophic Lateral Sclerosis. Biogen https://investors.biogen.com/news-releases/news-release-details/biogen-and-ionis-announce-toplinephase-1-study-results.
18. Brettschneider, J. et al. Stages of pTDP-43 pathology in amyotrophic lateral sclerosis. Ann. Neurol. 74, 20-38 (2013).
19. Shodai, A. et al. Aberrant assembly of RNA recognition motif 1 links to pathogenic conversion of TAR DNA-binding protein of 43 kDa (TDP-43). J. Biol. Chem. 288, 14886-14905 (2013).
20. Cox, A. D., Fesik, S. W., Kimmelman, A. C., Luo, J. & Der, C. J. Drugging the undruggable RAS: Mission possible? Nat. Rev. Drug Discov. 13, 828-851 (2014).
21. Tamaki, Y. et al. Elimination of TDP-43 inclusions linked to amyotrophic lateral sclerosis by a misfolding-specific intrabody with dual proteolytic signals. Sci. Rep. 8, 6030 (2018)
22. Mercier, J., Ruffin, M., Corvol, H. & Guillot, L. Gene Therapy: A Possible Alternative to CFTR Modulators? Front. Pharmacol. 12, 648203 (2021).
23. Teng, K. W. et al. Selective and noncovalent targeting of RAS mutants for inhibition and degradation. Nat. Commun. 12, 2656 (2021).
24 Pozzi, S. et al. Virus-mediated delivery of antibody targeting TAR DNA-binding protein-43 mitigates associated neuropathology. J Clin Invest 129 (4), 1581-1595 (2019).
25. Le, N. T. et al. Motor neuron disease, TDP-43 pathology, and memory deficits in mice expressing ALS-FTD-linked UBQLN2 mutations. Proc Natl Acad Sci USA 113 (47), E7580-E7589 (2016).
26. Girard-Gagnepain, A. et al. Baboon envelope pseudotyped LVs outperform VSV-G-LVs for gene transfer into early-cytokine-stimulated and resting HSCs. Blood (2014) 124 (8): 1221-1231.
27. Yokoo, H. et al. Cell-Penetrating Peptides: Emerging Tools for mRNA Delivery. Pharmaceutics 2022, 14, 78.
28. Chen, K. and D. Pei. Engineering Cell-Permeable Proteins through Insertion of Cell-Penetrating Motifs into Surface Loops. ACS Chem. Biol. 2020, 15, 9, 2568-2576.
29 Abels, E. R. et al. Glioblastoma-associated microglia reprogramming is mediated by functional transfer of extracellular miR-21. Cell Rep. 28, 3105-3119 (2019).
30. Cheng, L., Hill, A. F. Therapeutically harnessing extracellular vesicles. Nat Rev Drug Discov 21, 379-399 (2022). https://doi.org/10.1038/s41573-022-00410-w
31. Mangeot, P-E. et a. Protein Transfer Into Human Cells by VSV-G-induced Nanovesicles. Molecular Therapy 19 (9), 1656-1666 (2011).
32. Dixon, A. S. et al., NanoLuc Complementation Reporter Optimized for Accurate Measurement of Protein Interactions in Cells. ACS Chem Biol 11, 400-408 (2016).
33. Kowarz, E. et al, Optimized Sleeping Beauty transposons rapidly generate stable transgenic cell lines. Biotechnol J 10, 647-653 (2015).
34. Mates, L. et al., Molecular evolution of a novel hyperactive Sleeping Beauty transposase enables robust stable gene transfer in vertebrates. Nat Genet 41, 753-761 (2009).
35. Weber, K. et al. Lentiviral gene ontology (LeGO) vectors equipped with novel drug-selectable fluorescent proteins: new building blocks for cell marking and multi-gene analysis. Gene Ther 17, 511-520 (2010).
36. Lemaitre, C. et al. The HERV-K human endogenous retrovirus envelope protein antagonizes Tetherin antiviral activity. J Virol 88, 13626-13637 (2014).
37. Robinson-McCarthy, L. R. et al. Reconstruction of the cell entry pathway of an extinct virus. PLOS Pathog 14, e1007123 (2018).
38. Kingwell, K. et al. Base editors hit the clinic. Nat. Rev. Drug Discov. 21, 545-547 (2022).
39. St. Martin, A. et al. A fluorescent reporter for quantification and enrichment of DNA editing by APOBEC-Cas9 or cleavage by Cas9 in living cells. Nucleic Acids Res. 46, gky332-(2018).

1. An engineered FACT (fusogenic biovesicle-producing adoptive cell therapy) cell comprising (i) a polynucleotide sequence encoding one or more fusogens and/or proteins with cell penetrating properties, and (ii) a polynucleotide sequence serving as or encoding one or more cargo molecules.

2. The FACT cell of claim 1, additionally comprising a polynucleotide sequence encoding one or more gag-like proteins.

3. A method for delivering one or more cargo molecules to a target cell, comprising culturing a target cell with a FACT cell of claim 1 under conditions promoting delivery of a cargo molecule from the FACT cell to the target cell.

4. A method for delivering one or more cargo molecules to a target cell of a subject, comprising administering to a subject a population of FACT cells of claim 1.

5. A method for treating a disease or condition in a subject, comprising administering to a subject in need thereof a population of FACT cells of claim 1, wherein one or more cargo molecules are therapeutic molecules for the disease or condition.

6. The FACT cell of claim 1 wherein the one or more cargo molecules is a DNA molecule, RNA molecule, peptide or protein.

7. The FACT cell of claim 1 wherein the fusogen or protein with cell penetrating properties is encoded in a viral genome, in the human genome, or the genome of a non-human mammal, or the fusogen or protein with cell penetrating properties is a functional variant thereof.

8. The FACT cell of claim 1 wherein the fusogen or protein with cell penetrating properties is fully human.

9. The FACT cell of claim 1 wherein the fusogen or protein with cell penetrating properties has low or poor immunogenicity.

10. The FACT cell of claim 1 wherein the fusogen or protein with cell penetrating properties is syncytin A, or a functional variant thereof.

11. The FACT cell of claim 1 wherein the fusogen or protein with cell penetrating properties is HERV-K or a functional variant thereof.

12. The FACT cell of claim 2 wherein the gag-like protein is encoded in the human genome or the genome of a non-human mammal, or the gag-like protein is a functional variant thereof.

13. The FACT cell of claim 2 wherein the gag-like protein is human Arc (hArc) or PEG10, or a functional variant thereof.

14. The cell of claim 1 the cell may be a T cell, a monocyte, a megakaryocyte, a neuron, an epithelial cell, such as lung epithelial cells, or a stem cell.

15. The method of claim 4 wherein the subject is suffering amyotrophic lateral sclerosis, Alzheimer's disease or cancer.

16. A fusogenic biovesicle (BV) comprising (i) one or more fusogens or proteins with cell penetrating properties, (ii) one or more cargo molecules, and (iii) optionally one or more gag-like proteins.

17. The fusogenic BV of claim 16 wherein the BV is fully human

18. The fusogenic BV of claim 16 wherein the BV has low or poor immunogenicity.

19. A method for delivering one or more cargo molecules to a target cell, comprising culturing a target cell with a BV of claim 16 under conditions promoting delivery of a cargo molecule from the BV cell to the target cell.

20. The BV of claim 16 wherein the fusogen or protein with cell penetrating properties is encoded in a viral genome, in the human genome, or the genome of a non-human mammal, or the fusogen or protein with cell penetrating properties is a functional variant thereof.

21. The BV of claim 16 wherein the fusogen or protein with cell penetrating properties is syncytin A, or a functional variant thereof.

22. The BV of claim 16 wherein the fusogen or protein with cell penetrating properties is HERV-K or a functional variant thereof.

23. The BV of claim 16 wherein the gag-like protein is encoded in the human genome or the genome of a non-human mammal, or the gag-like protein is a functional variant thereof.

24. The BV of claim 16 wherein the gag-like protein is human Arc (hArc) or PEG10, or a functional variant thereof.

25. The BV of claim 16 wherein the one or more cargo molecules is a DNA molecule, RNA molecule, peptide or protein.

26. The FACT cell of claim 6, wherein the protein is a gene-editing enzyme.

27. The FACT cell of claim 26, wherein the gene-editing enzyme is a base editor, a prime editor, a TALE nuclease, or a CRISPR/Cas,zinc-finger nuclease.

28. The FACT cell of claim 27, wherein the base editor is one or more of an adenine and a cytosine base editor.

29. The BV of claim 25, wherein the protein is a gene-editing enzyme.

30. The BV of claim 29, wherein the gene-editing enzyme is a base editor, a prime editor, a TALE nuclease, or a CRISPR/Cas,zinc-finger nuclease.

31. The BV of claim 30, wherein the base editor is one or more of an adenine and a cytosine base editor.
